(19) United States
(12) Patent Application Publication
(10) Pub. No.: US 2007/0244437 A1
(43) Pub. Date: Oct. 18, 2007

(54) Fluid delivery system with bulk container and pump assembly

(76) Inventors: Luis Castillo, Allison Park, PA (US); James R. Neill, Oakdale, PA (US)

Correspondence Address: GREGORY L BRADLEY;MEDRAD INC, ONE MEDRAD DRIVE, INDIANOLA, PA, 15051, US

(21) Appl. No.: 11/396,376

(22) Filed: Mar. 31, 2006

(51) Int. Cl. A61M 1/00

(52) U.S. Cl. 604/151

(57) ABSTRACT

A fluid delivery system is disclosed including a patient interface device and a bulk container and pump assembly in fluid communication with the patient interface device for supplying a medical fluid to patient. The assembly includes a container body including a closed end and defining an opening leading to an interior chamber for holding the medical fluid, and a pump device closing and sealing the opening in the container body and adapted to draw the medical fluid from the container body and deliver the medical fluid to the patient. A method of supplying a medical fluid to a fluid delivery system is also disclosed and includes providing the bulk container and pump assembly and releasably associating the assembly with a drive device for driving the pump device. The assembly includes a pump device in the form of a gear pump including spur gears that are driven by the drive device.

### BACKGROUND OF THE INVENTION

1. Field of the Invention

The present invention relates to fluid delivery systems for supplying fluids to patients and, more specifically, to a fluid delivery system including a bulk container and pump assembly suitable for delivering a fluid to a patient under pressure.

2. Description of Related Art

In many medical procedures, it is desirable to inject a fluid into a patient. In certain medical procedures such as computed tomography (“CT”) and angiography, different types of contrast media (often referred to simply as contrast) are injected into a patient for diagnostic and therapeutic imaging purposes. For example, contrast media are used in diagnostic X-ray procedures such as angiography, venography, and urography, CT scanning, magnetic resonance imaging (MRI), and ultrasonic imaging. Contrast media are also used during therapeutic procedures including, for example, angioplasty and other interventional radiological procedures. Often, the medical procedure may involve the supply of intravenous drugs, replacement fluids such as saline and glucose, and the like.

A number of problems arise in the use of current fluid delivery systems and methods for injecting fluid into a patient's body. For example, it is often difficult to accurately control the pressure and flow rate of the fluid delivered by the pumping mechanism. Although peristaltic pumps have long been successfully used in relatively low pressure applications, peristaltic pumps are difficult to control with accuracy.

In the case of relatively higher pressure applications, such as CT and angiography, mechanized syringe injectors are used. The use of mechanized syringe injectors also results in a number of drawbacks. Current mechanisms for powering and controlling syringe pumps are complicated, inefficient, and costly. Expensive and bulky pressure jackets for housing the syringe pumps are often required to prevent failure at high pressures. Syringe pumps are severely limited in that only the volume of fluid contained in the syringe may be injected at one time. Disposable syringe pumps are costly. Moreover, the rise time of syringe injectors is limited by the inertia of the extensive drive train required to translate motor rotation into syringe plunger motion.

These and other drawbacks in current syringe pumping systems create and magnify a number of inefficiencies in current procedures for injecting contrast media. For example, a number of factors, including, but not limited to, the procedure to be performed and the size of the patient, determine: (i) the contrast to be used, (ii) the concentration thereof, and (iii) the amount to be injected. Under current practice of injecting contrast media via syringe pumping systems, hospitals must purchase and stock many contrast media concentrations in multiple container sizes in an attempt to provide the correct concentration and amount of a specific contrast for a specific procedure, while minimizing the wastage of expensive contrast. This is likewise the case with more routinely used fluids, such as saline or glucose concentrations, used in hospital or other patient-care settings.

Thus, most contrast media are provided by manufacturers in numerous concentrations in sterilized containers (such as glass bottles or plastic packages) ranging incrementally in size from 20 ml to 200 ml. The contrast is generally aspirated from such containers via the syringe pump used to inject the contrast, and any contrast remaining in the container is discarded to prevent infection with potentially contaminated contrast. The hospital staff is faced with the task of choosing an appropriately sized contrast container to assure an optimum study while minimizing discarded contrast. Time-consuming procedures are required to reload the syringe if more contrast is required than originally calculated. The inventory of contrast containers required under the current system increases costs and regulatory burdens throughout the contrast media supplier-consumer chain.

Beyond the field of contrast media supplied for radiological studies and therapeutic procedures, fluids that are more routinely supplied to patients in hospital settings, such as glucose and saline concentrations, are typically provided in glass or plastic bottles or in IV bags of varying size and concentration. The contents of such containers are emptied, for example, under the force of gravity or by the attachment of a pump device to the container to inject the contents under pressure into a patient, such as may be desirable for the intravenous delivery of drugs to a patient. Such extra pump devices are typically complicated, costly, and bulky devices that require significant up-front capital expenditures and require some degree of expertise on part of the medical practitioner charged with setting-up and priming the pump device. The time spent configuring such pump devices to operate properly takes away from the care time that may be administered to a patient in a hospital or other patient care setting. An intent of the invention described herein is to provide a low cost and user-friendly alternative to such pump devices and a fluid delivery system that may be generally used for many different fluid delivery procedures.

### SUMMARY OF THE INVENTION

In one aspect, the invention is directed to a bulk container and pump assembly generally comprising a container body comprising a generally closed end and defining an opening leading to an interior chamber within the container body for holding a medical fluid, and a pump device closing and sealing the opening in the container body and adapted to draw the medical fluid from the container body and deliver the medical fluid to a patient. The container body may be substantially rigid, or take the form of containers with soft sides and pliable containers such as IV bags each with an appropriate opening for receiving the pump device. The container body and pump device may be conveniently disposable as a singular unit.

In one form, the pump device may be a gear pump. Such a gear pump may be disposed in a housing sealing the opening in the container body and comprise spur gears. The spur gears may be disposed in the housing sealing the opening in the container body.

The pump device may comprise a housing sealing the opening in the container body, with the interior of the housing in fluid communication with the interior chamber defined by the container body. The housing may comprise a valve for dispensing the medical fluid from the container body via the housing to the patient.

Another feature of the assembly relates to an encoding device which may be associated with the container body. The encoding device may be operable to provide container body information to a sensor. The encoding device may be an optically or a mechanically readable device, as examples. The container body information may include, but is not limited to, container body volume information and the type of medical fluid contained in the container body.

In another aspect, the invention is directed to a fluid delivery system comprising a patient interface device and a bulk container and pump assembly in fluid communication with the patient interface device for supplying a medical fluid to patient via the patient interface device. The assembly includes a container body comprising a generally closed end and defining an opening leading to an interior chamber within the container body for holding a medical fluid, and a pump device closing and sealing the opening in the container body and adapted to draw the medical fluid from the container body and deliver the medical fluid to the patient. As indicated hereinabove, the container body may be substantially rigid, or take the form of containers with soft sides and pliable containers such as IV bags each with an appropriate opening for receiving the pump device. The container body and pump device may be conveniently disposable as a singular unit.

In one form, the pump device may be a gear pump. Such a gear pump may be disposed in a housing sealing the opening in the container body and comprise spur gears. The spur gears may be disposed in the housing sealing the opening in the container body.

The pump device may comprise a housing sealing the opening in the container body, with the interior of the housing in fluid communication with the interior chamber defined by the container body. The housing may comprise a valve for dispensing the medical fluid from the container body via the housing to the patient. A plunger may be disposed in the container body for dispensing the medical fluid from the container body, for example, upon actuation of the pump device.

An encoding device may be associated with the container body. The encoding device may be operable to provide container body information to a sensor. The encoding device may be an optically or mechanically readable device. The container body information may include, but is not limited to, container body volume information and the type of medical fluid contained in the container body.

A drive device may be operatively connected to the pump device for driving the pump device. As indicated, in one form, the pump device may be a gear pump and the drive device may be adapted to drive the gear pump. The gear pump may comprise spur gears and the drive device may include a motor coupled to the spur gears for driving the spur gears.

A fluid level sensor may be associated with the container body for determining the level of medical fluid in the container body. Such a fluid level sensor may be adapted to sense when the medical fluid reaches a minimum level in the container body.

The bulk container and pump assembly may be releasably lockable to a base supporting the drive device adapted to drive the pump device. The bulk container and pump assembly may be releasably lockable to the base with one or more releasable clamps. A sensor may be associated with the releasable clamp. Such a sensor may be adapted to identify a locked position of the releasable clamp.

In another embodiment, the fluid delivery system generally comprises a control unit, a drive device operatively controlled by the control unit, and a bulk container and pump assembly. As in previous embodiments, the assembly includes a container body comprising a generally closed end and defining an opening leading to an interior chamber within the container body for holding a medical fluid, and a pump device closing and sealing the opening in the container body and adapted to draw the medical fluid from the container body and deliver the medical fluid to the patient. The drive device is operatively connected to the pump device for driving the pump device.

A fluid level sensor may be associated with the container body for determining the level of medical fluid in the container body. The fluid level sensor may be adapted, for example, to sense when the medical fluid reaches a minimum level in the container body and send a signal to the control unit.

In the fluid delivery system, a sensor may be associated with a releasable clamp adapted to releasably lock the bulk container and pump assembly to a base supporting the drive device. The sensor may be adapted to identify a locked position of the releasable clamp and send a signal to the control unit.

An encoding device may be associated with the container body. The encoding device may be operable to provide container body information to a sensor operatively connected to the control device. In form, encoding device may be an optically readable device.

A further aspect of the invention relates to a method of supplying a medical fluid to a fluid delivery system, generally comprising providing a bulk container and pump assembly, and releasably associating the bulk container and pump assembly with a drive device for driving a pump device of the assembly. As indicated hereinabove, the assembly may comprise a container body comprising a generally closed end and defining an opening leading to an interior chamber within the container body for holding a medical fluid, and a pump device closing and sealing the opening in the container body and adapted to draw the medical fluid from the container body.

Other aspects of the method may include releasably locking the bulk container and pump assembly to a base supporting the drive device, and sensing a locked state of the bulk container and pump assembly with the base.

In the method, actuation of the drive device may drive the pump device of the bulk container and pump assembly to draw the medical fluid from the container body. The drive device may be actuated by a control unit controlling operation of the drive device. The medical fluid in the container body may be continuously monitored for a minimum level of medical fluid in the container body, for example, during the operation of the pump device in the assembly.

Further, the container body may comprise an encoding device operable to provide container body information to a sensor, and the method may further comprise sensing the encoding device with the sensor. The sensed container body information may be provided to a control unit which desirably modifies operation of the drive device based on the sensed container body information. In one embodiment, the encoding device may be sensed by an optical sensor, and the container body information may comprise container body volume information and the type of medical fluid contained in the container body, as well as other information concerning the container body, its contents, or the pump device as desired.

Further details and advantages of the invention will become clear upon reading the following detailed description in conjunction with the accompanying drawing figures.

### BRIEF DESCRIPTION OF THE DRAWINGS

FIG. 1 is perspective view of a bulk container and pump assembly in accordance with an embodiment of the invention.

FIG. 2 is an exploded perspective view of the bulk container and pump assembly shown in FIG. 1.

FIG. 3 is a perspective view of the bulk container and pump assembly shown in FIG. 1, further showing a drive device for imparting motion to a pump device in the assembly.

FIG. 4 is a side view of the bulk container and pump assembly and the drive device shown in FIG. 3.

FIG. 5 is an exploded perspective view of the drive device shown in FIG. 3, further showing the bulk container and pump assembly associated with the drive device.

FIG. 6 is perspective view of the bulk container and pump assembly and associated drive device incorporated into a fluid delivery system in accordance with another aspect of the invention.

### DESCRIPTION OF THE PREFERRED EMBODIMENTS

For purposes of the description hereinafter, spatial orientation terms, if used, shall relate to an embodiment of the invention as it is oriented in the accompanying drawing figures or otherwise described in the following description of the invention. However, it is to be understood that the embodiments described hereinafter may assume many alternative variations and configurations. It is also to be understood that the specific devices illustrated in the accompanying drawing figures and described herein are simply exemplary to the invention and should not be considered as limiting.

One aspect of the invention is directed to a bulk container and pump assembly. Another aspect of the invention incorporates the bulk container and pump assembly as part of a fluid delivery system. In the system, a pump draws fluid from a bulk container and then delivers the fluid to a patient through an appropriate interface device such as a catheter, IV needle, and/or tubing set. The system may be adapted to supply a relatively small volume of fluid to a patient allowing the system to be used as a drug infusion, gene therapy, or chemotherapy vehicle, or a larger volume of fluid such as contrast media typically used during computed tomography (“CT”) and angiographic or other cardiovascular procedures. The bulk container and pump assembly is desirably constructed as a composite device from relatively inexpensive materials. This advantageously allows the bulk container and pump assembly to be disposed of as a singular unit after a single use thereby increasing patient turn around time due to less user interface with the fluid delivery system, and decreasing the possibility of system and medical fluid contamination.

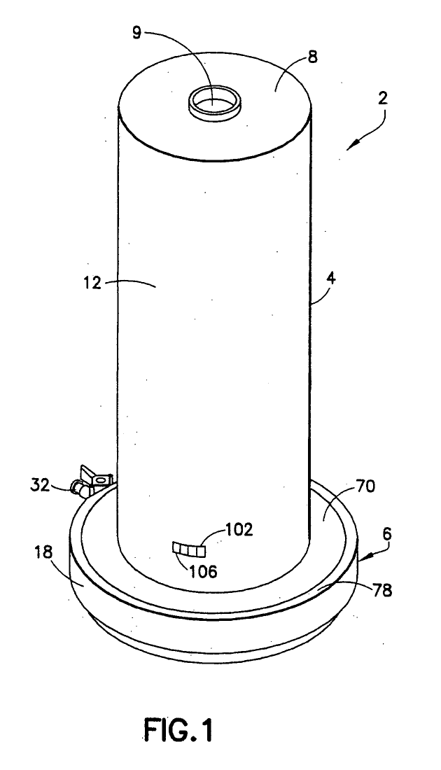

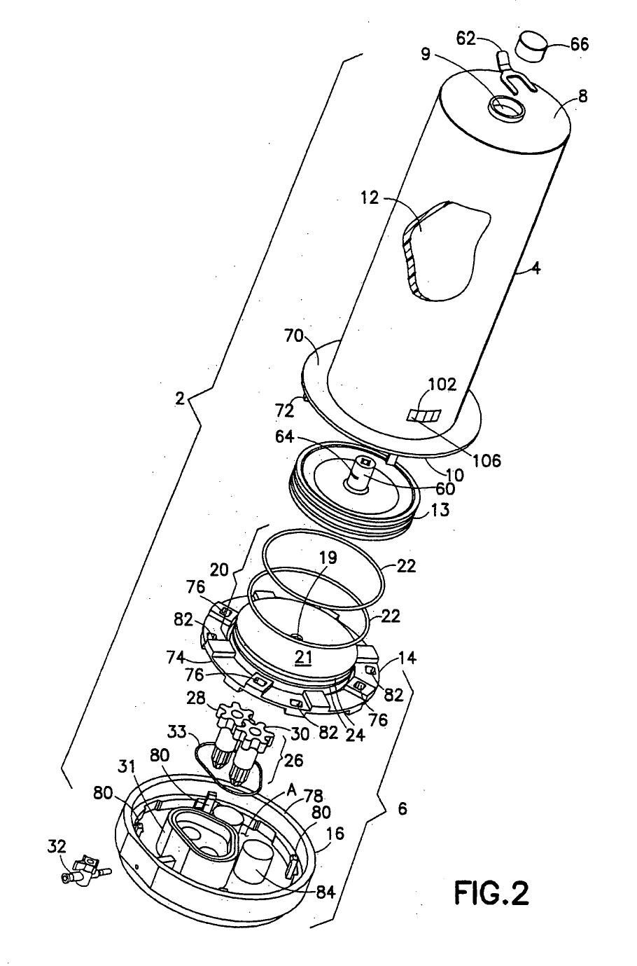

With reference to FIGS. 1 and 2, a bulk container and pump assembly, denoted generally as reference numeral 2 (hereinafter “assembly 2”), generally comprises a container body 4 and a pump device 6. Container body 4 defines a substantially closed top end 8 defining an opening 9, and further defines a bottom opening 10 leading to an interior chamber 12 defined within container body 4 for holding a medical fluid. The medical fluid may be contrast medium for use in medical imaging applications, a fluid for use in drug infusions, a fluid for use in gene therapy or chemotherapy, or a general medical fluid used in hospital or other patient care settings such as saline and glucose IV's. Container body 4 is substantially rigid and may include, but is not limited to, rigid containers, containers with soft sides, and pliable containers such as IV bags.

Container body 4 also includes a plunger 13 positioned within interior chamber 12. As indicated, opening 9 is defined in generally closed end 8. Opening 9 allows for the creation of a pressure differential within interior chamber 12 used to empty the fluid from interior chamber 12 as will be discussed in further detail hereinafter. Instead of an opening 9 in the closed end 8 of container body 4, a check valve or other structure that allows air, but not liquid to pass into interior chamber 12 may be utilized. Plunger 13 includes a plunger stem 60 which extends through opening 9 in a pre-use state of assembly 2. A plunger locking clip 62 is positioned over opening 9 and in engagement with plunger stem 60 extending from plunger 13 to secure the plunger stem 60 in place within opening 9. Plunger stem 60 extends through opening 9 in a pre-use state of assembly 2 to prevent displacement of plunger 13 before use and, further, desirably to seal opening 9 prior to use. Therefore, plunger stem 60 may operate as a plug for opening 9 in the pre-use state of assembly 2 and may be held in place by engagement of the plunger locking clip 62 with side grooves 64 defined in the plunger stem 60. If desired, an end cap 66 may be disposed at the end of plunger stem 60 to secure the disposition of plunger stem 60 in opening 9 and prevent the accidental dislodgement of plunger locking clip 62 from the plunger stem 60. Plunger stem 60, plunger locking clip 62, and end cap 66 operate as a removable sealing structure which allows the interior chamber 12 of container body 4 to be vented to atmospheric pressure and thereby allow pump device 6 to empty the contents of the container body 4 when actuated.

Pump device 6 generally closes and seals opening 10 in container body 4 and is adapted to draw fluid from container body 4 and deliver the fluid to a patient. Pump device 6 includes an upper housing body 14 and a lower housing body 16 generally forming a pump housing 18. Pump housing 18 is secured to container body 4 desirably via a snap-fit connection thereby sealing bottom opening 10 in container body 4. The interior of pump housing 18 is in fluid communication with interior chamber 12 defined by container body 4 via a discharge opening 19 in upper housing body 14 which is further configured for at least partial insertion into bottom opening 10 in container body 4, as discussed herein. In order to ensure that a fluid tight seal is provided between bottom opening 10 and upper housing body 14, a perimeter seal structure 20 may be associated with the upper housing body 14. Such a perimeter seal structure 20 may extend around a central cylindrical area 21 formed on upper housing body 14 which is adapted for insertion into bottom opening 10 defined in container body 4. As shown in FIG. 2, discharge opening 19 is defined in central cylindrical area 21. Seal structure 20 forms a seal between the interior wall of container body 4 defining bottom opening 10 and the central cylindrical area 21. In one form, the perimeter seal structure 20 may be formed by a pair of O-rings 22 which are disposed within perimetrical groves 24 extending around central cylindrical area 21 of upper housing body 14. As is known, O-rings are conventional structures that are used to seal fluid connections. In pump housing 18, O-rings 22 form a generally fluid tight barrier between the interior wall of container body 4 defining bottom opening 10 and the central cylindrical area 21 of upper housing body 14 when the upper housing body 14 is secured to the container body 4.

To secure the sealing of opening 10 in container body 4 by upper housing body 14, a securing connection is established between the container body 4 and pump housing 18. For ease of assembly, such a securing connection is desirably a snap-fit connection. For this purpose, container body 4 may be formed with a bottom flange 70 having depending engagement tabs 72. Upper housing body 14 is formed with an opposing and generally cooperating flange 74 which defines a plurality of receiving openings 76 that are adapted to accept the depending tabs 72 on bottom flange 70 of container body 4. The depending engagement tabs 72 may be conventional arrow-shaped or barbed tabs which engage the receiving openings 76 in a snap-fit manner and thereafter maintaining the engagement of bottom flange 70 with cooperating flange 74.

Lower housing body 16 is a generally bowl-shaped structure with an upstanding lip or rim 78 defining an interior volume or area A in which the upper housing body 14 is secured. Lower housing body 16 includes a plurality of internal engagement tabs 80 which are adapted to engage a second series of receiving openings 82 defined in cooperating flange 74 in a similar manner to container body 4 and upper housing body 14 just described. Again, the internal engagement tabs 80 may be conventional arrow-shaped or barbed tabs which engage the receiving openings 82 in a snap-fit manner and thereafter maintain the engagement of upper housing body 14 with lower housing body 16. While a snap-fit connection is indicated as a means of securing container body 4 to upper housing body 14 and securing upper housing body 14 to lower housing body 16, this exemplary connection technique should not to be construed as limiting the possible additional alternative methods which may be used to secure the connection of pump housing 18 to container body 4. One example of such an alternative means includes the use of an adhesive, typically a medical grade adhesive, to form a securing connection between bottom flange 70 on container body 4 and cooperating flange 74 of upper housing body 14 and, analogously, to form a sealing connection between cooperating flange 74 and the interior of lower housing body 16. In the former securing connection, an adhesive may be used to secure bottom flange 70 to cooperating flange 74. In the latter securing connection, an adhesive may be used to secure cooperating flange 74 to the interior of lower housing body 16. Another example is the use of mechanical fasteners in place of depending engagement tabs 72 and projecting engagement tabs 80. A further example is the use of simple mechanical clamping connections between bottom flange 70 and cooperating flange 74 and between the cooperating flange 74 and the interior of lower housing body 16. As further shown in FIG. 2, spacers 84 may be formed as part of lower housing body 16. The spacers 84 are generally adapted to engage or contact the bottom interior surface of central cylindrical area 21 formed on upper housing body 14 and stabilize the connection between the upper housing body 14 and lower housing body 16.

Pump device 6 includes a gear pump 26 enclosed within pump housing 18 between upper housing body 14 and lower housing body 16. Gear pump 26 is capable of delivering accurate volumes of fluid at substantially uniform pressures without or with a minimum of pressure pulsations as is common in the syringe-type injectors discussed previously. Gear pump 26 comprises a first spur gear 28 that drives an identical second spur gear 30. However, both the first and second spur gears 28, 30 may be driven in which case the first and second spur gears 28, 30 rotate in opposite directions. In operation, the equal and opposite motions of the first spur gear 28 and the second spur gear 30 creates vacuum pressure within interior chamber 12 of container body 4 which pulls plunger 13 downward within the interior chamber 12 thereby forcing fluid from the container body 4 into pump housing 18 via discharge opening 19 in upper housing body 14. Desirably, the first and second spur gears 28, 30 forming gear pump 26 are disposed within an internal housing 31 formed as part of lower housing body 16 of pump housing 18. The internal housing 31 is in fluid communication with the interior chamber 12 of container body 4 via discharge opening 19 in upper housing body 14. As gear pump 26 operates, fluid contained in interior chamber 12 of container body 4 is pulled through discharge opening 19 and into internal housing 31. An outlet valve 32 is positioned on lower housing body 16 of pump device 6 which is fluidly connected to internal housing 31 for dispensing fluid from pump device 6 and allows the fluid drawn from container body 4 to exit assembly 2. Outlet valve 32 may be a conventional stopcock valve with a standard male or female luer end connector.

As shown in FIG. 2, an O-ring 33 may be provided to seal internal housing 31 from the interior area A generally defined by lower housing body 16. The sealing O-ring 33 is positioned to seal against the bottom interior surface of central cylindrical area 21 formed on upper housing body 14 and around discharge opening 19 therein. Once internal housing 31 is sealed to the central cylindrical area 21, which will occur automatically with the securing of upper housing body 14 to lower housing body 16, a sealed fluid path is provided from the interior chamber 12 of container body 4 to the interior of internal housing 31 and, ultimately, to outlet valve 32.

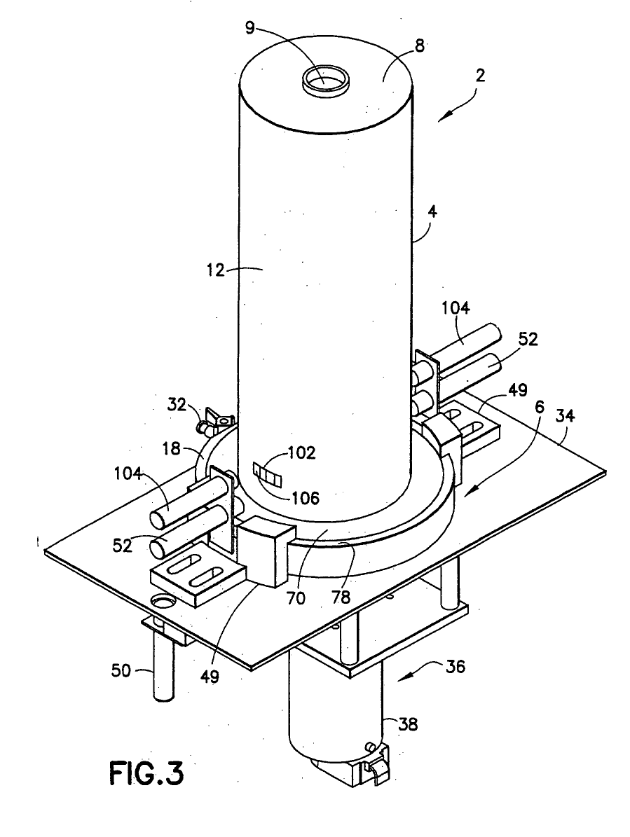

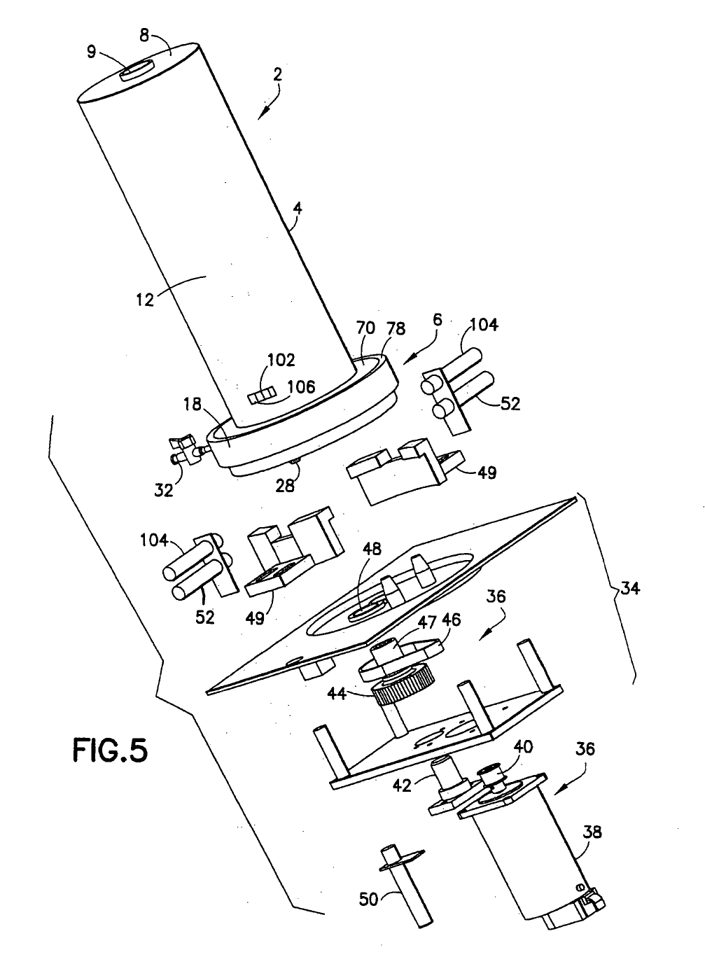

With further reference to FIGS. 3-5, a base interface structure or platform 34 is adapted to support the bulk container and pump assembly 2 and, further, supports devices that operate and monitor assembly 2. In the embodiment depicted, a drive device 36 is supported on base 34 desirably in a convenient position below pump device 6 of assembly 2 for driving pump device 6. The depicted drive device 36 includes a motor 38, a drive train pulley 40, a drive train spindle 42, a gear pump pulley 44, and a drive train belt 46. Motor 38 is operatively coupled to, and drives, drive train pulley 40. Drive train pulley 40 is operatively coupled to drive train spindle 42 via gear pump pulley 44 and drive train belt 46. The motive power produced by motor 38 is applied to drive train belt 46 which is used to rotate gear pump pulley 44. Drive train belt 46 is tensioned between gear pump pulley 44 and drive train pulley 40. Gear pump pulley 44 is coupled to first spur gear 28 to drive the first spur gear 28, which drives second spur gear 30 due to the meshed engagement of the first spur gear 28 with the second spur gear 30. As shown in FIG. 5, gear pump pulley 44 may include a shaft 47 that extends through an opening 48 in base 34 to engage the first spur gear 28 of gear pump 26. In this manner, gear pump pulley 44 is automatically operatively connected to gear pump 26 when assembly 2 is associated with base 34. Once gear pump pulley 44 is coupled to the first spur gear 28 of gear pump 26, the drive device 36 may be actuated to drive spur gears 28, 30 of gear pump 26 and dispense fluid from container body 4. Motor 38 may be a DC motor or any other suitable motor. The configuration of drive device 36 in FIGS. 3-5 is provided as an exemplary embodiment of a device which may used to drive pump device 6. Any suitable mechanically equivalent drive arrangement may be used in place of the pulley-drive belt system shown. For example, drive train pulley 40 may be a conventional spur gear that drives a spur gear provided in place of gear pump pulley 44.

At least one, and desirably two, releasable clamps 49 are disposed on base 34 for locking assembly 2 to base 34 and, further, for operatively interfacing pump device 6 with drive device 36. Clamps 49 are desirably adapted to operate in unison, either mechanically or electrically, to engage assembly 2 to secure the assembly 2 to base 34. A clamp position sensor 50 is positioned on base 34 and is associated with one of the clamps 49. Clamp position sensor 50 is adapted to identify the position of clamps 49 and, more particularly, identify when the clamps 49 are in a locked position. Clamps 49 are generally configured to engage onto and desirably at least partially over rim 78 formed on lower housing body 16. The rim 78 on lower housing body 16, as discussed previously, generally defines an interior volume or area A wherein internal housing 31 is provided which encloses gear pump 26. The bottom flange 70 formed on container body 4 is disposed within rim 78, and upper housing body 14 is secured to lower housing body 16 within interior area A.

Clamp position sensor 50 is adapted to identify when clamps 49 are in a locked position engaging the rim 78 on lower housing body 16 of pump housing 18 (and possibly lower flange 70 of container body 4) which indicates the presence of a bulk container and pump assembly 2 on base 34. Clamp position sensor 50 desirably sends a signal to drive device 36 or a control unit associated with drive device 36 that prevents the drive device 36 from activating unless clamps 49 are in a locked position and, thus, assembly 2 is properly engaged with the drive device 36. Clamp position sensor 50 may be an optical sensor, or a simple electromechanical switch that is triggered by the locking or unlocking movement of one or both of clamps 49. An optical embodiment is shown for clamp position sensor 50 in FIGS. 3-6.

Base 34 also supports at least one, and desirably two, fluid level sensors 52 for determining the level of fluid in container body 4. Fluid level sensors 52 are particularly adapted to determine when the fluid reaches a minimum level in container body 4 during operation of assembly 2. Once fluid level sensors 52 determine that the fluid in container body 4 has reached a pre-identified minimum level, the fluid level sensors 52 sends an interrupt signal to drive device 36 or to a control unit associated with the drive device 36 which causes the drive device 36 to cease operation. This interrupt or stop signal thereby prevents the system from delivering air bubbles to a patient. Fluid level sensors 52 may be optical sensors, ultrasonic sensors, acoustic sensors or any other suitable sensors known in the medical field for detecting the presence of air bubbles in plastic or glass containers or medical tubing. Fluid level sensors 52 may be conveniently supported on the structure of releasable clamps 49 as illustrated.

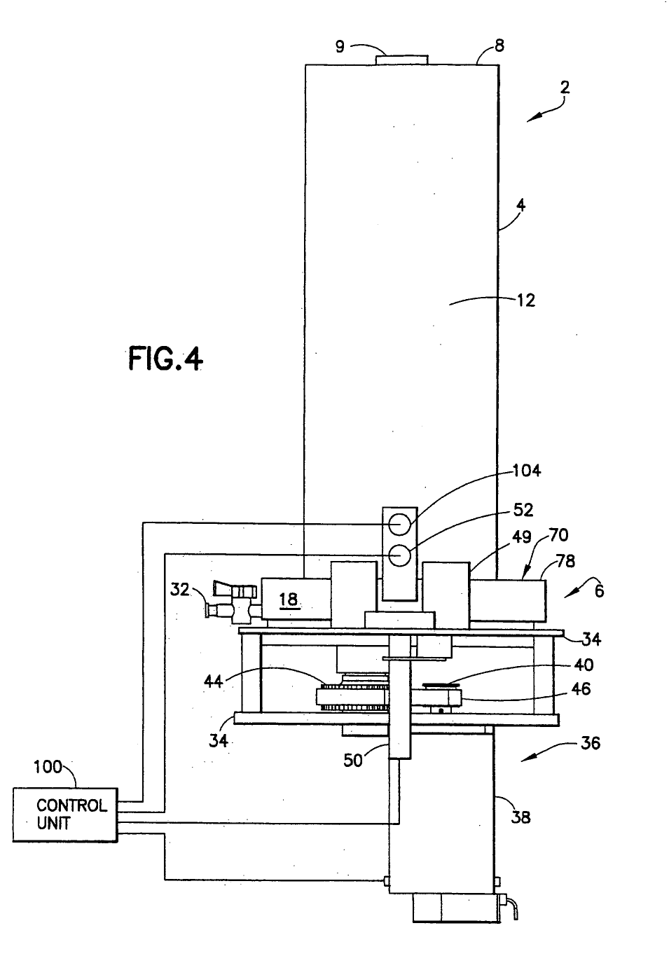

As generally described previously, fluid level sensors 52 and clamp position sensor 50 may be operatively connected to drive device 36 or to a control unit. An exemplary control unit 100 is shown schematically in FIG. 4 and is operatively associated with the drive device 36. Control unit 100 may be an external computer system or like device. Control unit 100 may also be used to control operation of assembly 2 when associated with drive device 36 by controlling drive device 36. Desirably, control unit 100 operates drive device 36 in accordance with parameters associated with the size (i.e., volume) of container body 4 and, further, the specific contents container body 4, as well as other parameters. For this purpose, one or more encoding devices 102 are disposed on the container body 4 that are read by one or more container body sensors 104 which may be conveniently placed in proximity to fluid level sensors 52, for example, above fluid level sensors 52. For example, the supporting structure for fluid level sensors 52 may additionally be used to support container body sensors 104. Container body sensors 104 are used to read the encoding devices 102 on container body 4 and send a signal or signals to control unit 100 concerning the size or contents, or both, of assembly 2 associated with drive device 36, or other information relating to assembly 2 as described herein. As a result, control unit 100 is able to detect both the presence of assembly 2 on base 34 via clamp position sensor 50, but is also able to “read” assembly 2 to obtain information concerning assembly 2. This information may be used as programming inputs to the control unit 100 which will thereafter direct operation of drive device 36 (or modify operation thereof) and, hence, control the discharge of fluid from container body 4. Thus, a specific fluid delivery/injection procedure or “protocol” tailored to the patient may be performed in accordance with the information detected or “read” from container body 4 and transmitted to control unit 100.

In this instance, encoding device 102 on container body 4 is a series of indented (i.e., recessed) spaced bars 106 of varying indentation provided in the container body 4. Container body 4 is desirably formed of clear or slightly opaque molded plastic material to allow the spaced bars 106 to be read by optical means. The corresponding “reading” container body sensors 104 are positioned to read spaced bars 106 when the assembly 2 is mounted to base 34. For this purpose, base 34 may be configured so that assembly 2 may be mounted to base 34 in a manner such that spaced bars 104 are always properly associated with container body sensors 104. For example, base 34 may be formed with an alignment groove (not shown) and the lower housing body 16 of pump housing 18 may be formed with a depending tab (not shown) for engaging the alignment groove which will prevent assembly 2 from being clamped to base 34 in any other orientation other than a “correct” orientation, wherein encoding device 102 is aligned with container body sensors 104. This configuration may be further used to assure proper operative engagement between pump device 6 and drive device 36.

Based on the foregoing, when assembly 2 is properly mounted and clamped to base 34, pump device 6 is automatically associated with drive device 36 and container body sensors 104 may “read” the encoding devices 102 and forward a signal or signals to the control unit 100. Control unit 100 desirably recognizes the presence of assembly 2 from clamp position sensor 50 and, further, the signals from container body sensors 104. Control unit 100 then interprets the information contained in the signals from container body sensors 104 and conducts operation of drive device 36 (or modifies operation thereof) in accordance with the control information contained in the signal(s) from container body sensors 104. Examples of information which could be encoded in encoding device 102 include dimensions (i.e., volume) of the container body 4, suggested flow rate information for dispensing the contents of container body 4, and the fluid contained in container body 4. Other information that may be encoded in encoding devices 102 includes manufacturing information such as lot numbers, dates, and tool cavity number. As an alternative to encoding devices 102 being a series of indented spaced bars 106, the encoding devices 102 could also include raised surfaces corresponding to spaced bars 106 or a simple bar code. The encoding devices 102 could also be a mechanically read device, such as a slot, hole, or projection extending from container body 4 which registers with a switch or other electromechanical structure provided in place of sensors 104. Another alternative is to provide encoding devices 102 as optically readable devices, such as characters, dots, and other geometric shapes that may be read by an optical sensor, such as sensors 104. Further, the encoding devices 102 may contain different types of information and the container body sensors 104 may be separately connected to the control device 100 to provide separate input signals to the control device 100. While control unit 100 is shown physically separate from assembly 2 and drive device 36, the control unit 100 may be physically supported by base 34 and form a part of the overall system. Moreover, as indicated previously, fluid level sensors 52 are desirably operatively connected to control unit 100 and the fluid level signal generated by sensors 52 may be inputs to control unit 100. Control unit 100 may generate the interrupt signal to drive device 36 when a critical low level is identified by fluid level sensors 52 which causes the drive device 36 to cease operation thereby preventing the injection of air bubbles into a patient.

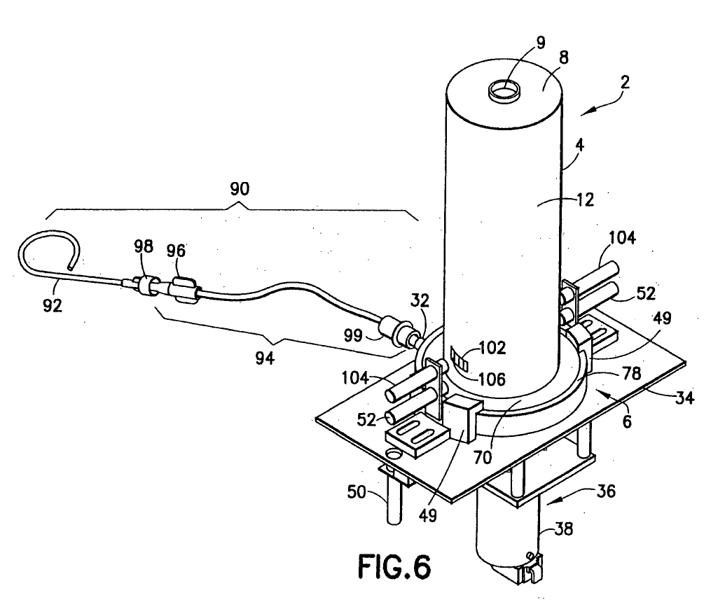

FIG. 6 shows the bulk container and pump assembly 2 associated with a patient interface device 90. Patient interface device or section 90 is used to connect the flow path of assembly 2 intravenously to a patient, and may include for this purpose a catheter 92 or other device for injecting fluid intravenously into patient such as an IV needle cannula (not shown). The patient interface device 90 is adapted for connection to outlet valve 32 of bulk container and pump assembly 2. The patient interface device 90 may include, for example, a medical tubing connection section 94 including a terminal or distal end connector 96 adapted to engage a luer connector 98 on catheter 92 and a proximal end connector 99 adapted to engage the luer connector element on outlet valve 32 of bulk container and pump assembly 2. A suitable catheter for catheter 92 and a suitable medical tubing connection section for section 94 may be found in U.S. patent application Ser. No. 60/741,146, filed Dec. 1, 2005 and entitled “Fluid Deliver System, Fluid Path, and Medical Connector for Use with the Fluid Deliver System and Fluid Path”. The disclosure of U.S. patent application Ser. No. 60/741,146 is incorporated herein by reference in its entirety. While the bulk container and pump assembly 2 was described hereinabove as having a single outlet valve 32 or port, a plurality of outlet valves 32 may be provided, if desired.

In operation, bulk container and pump assembly 2 is positioned on base 34 such that pump device 6 engages with drive device 36. The assembly 2 is locked in place on base 34 using releasable clamps 49. Clamp position sensor 50 sends a signal to drive device 36 or control unit 100 when the assembly 2 is properly locked in place on base 34 via clamps 49. The operator then removes end cap 66 from plunger stem 60 and removes the locking clip 62 from the plunger stem 60. The interior chamber 12 of container body 4 is now vented to atmospheric pressure. Fluid pressure in the container body 4 is typically sufficient to aspirate air from patient interface section 90 once outlet valve 32 is opened. However, catheter 92 or an equivalent IV needle in the patient interface section 90 is typically prepositioned within the vein or artery of a patient and, once the connection section 92 of patient interface section 90 is aspirated of air, the connection section 92 is connected to the prepositioned catheter 90. The assembly 2 is now desirably in fluid communication with the patient's vein or artery. The drive device 36 may be configured to start manually, for example, by a healthcare professional pushing a start button connected to the drive device 36, or the control unit 100 may be accessed to operate drive device 36 in a preprogrammed manner. For example, the control unit 100 may include an interface device such as a touch screen for inputting fluid injection protocol information into the memory of control unit 100 and then the control unit 100 may be accessed to start the injection procedure, for example, by touching a “start” button on the touch screen. Another interface device that may be provided is a handheld controller that starts the drive device 36 either directly or through inputs provided to the control device 100. Such a handheld controller may be adapted to control operation of control device 100 to control the fluid flow from container body 4.

Once drive device 36 is actuated, the drive device 36 drives spur gears 28, 30 of gear pump 26. The equal and opposite motions of spur gears 28, 30 creates vacuum pressure in the interior chamber 12 of container body 4 thereby pulling plunger 13 downward in interior chamber 12. The fluid in container body 4 is displaced into pump housing 18 and, in particular, into the internal chamber 31 in the lower housing body 16, and then through outlet valve 32 to the patient interface device 90 thereby delivering the fluid intravenously to a patient. Once fluid level sensors 52 determine that the fluid has reached a minimum level in container body 4, these sensors 52 send a cut-off or interrupt signal to drive device 36 or control unit 100. This signal or signals stops drive device 36 (either directly or through control unit 100), thereby preventing the injection of air bubbles into the patient. The attending healthcare professional may also stop the drive device 36 at any time using a handheld controller (not shown), if desired or by touching an appropriate “stop” button on a touch screen associated with control unit 100. Once the delivery of a fluid has stopped, the releasable clamps 48 may be released and the bulk container and pump assembly 2 is removed from base 34 and discarded as a singular unit. The base 34 and drive device 36 having remained fluid-isolated from assembly 2 may accept a new prefilled assembly 2 for use with another patient without fear of contamination to the new patient.

While the present invention was described by way of a detailed description of several embodiments of a fluid delivery system including one or more embodiments of a bulk container and pump assembly, those skilled in the art may make modifications and alterations to this invention without departing from the scope and spirit of the invention. Accordingly, the foregoing description is intended to be illustrative rather than restrictive. The invention described hereinabove is defined by the appended claims, and all changes to the invention that fall within the meaning and the range of equivalency of the claims are embraced within their scope.

The invention claimed is:

1. A bulk container and pump assembly, comprising:
a container body comprising a generally closed end and defining an opening leading to an interior chamber within the container body for holding a medical fluid; and
a pump device closing and sealing the opening in the container body and adapted to draw the medical fluid from the container body and deliver the medical fluid to a patient.

2. A bulk container and pump assembly as claimed in claim 1 wherein the container body is substantially rigid.

3. A bulk container and pump assembly as claimed in claim 1 wherein the pump device comprises a gear pump.

4. A bulk container and pump assembly as claimed in claim 3 wherein the gear pump is disposed in a housing sealing the opening in the container body.

5. A bulk container and pump assembly as claimed in claim 3 wherein the gear pump comprises spur gears.

6. A bulk container and pump assembly as claimed in claim 1 further comprising an encoding device associated with the container body and operable to provide container body information to a sensor.

7. A bulk container and pump assembly as claimed in claim 6 wherein the encoding device comprises an optically readable device.

8. A pump cassette as claimed in claim 6 wherein the container body information comprises at least container body volume information and the type of medical fluid contained in the container body.

9. A bulk container and pump assembly as claimed in claim 1, wherein the container body and pump device are disposable as a singular unit.

10. A fluid delivery system, comprising:
a patient interface device; and
a bulk container and pump assembly in fluid communication with the patient interface device for supplying a medical fluid to patient via the patient interface device, and comprising:
a container body comprising a generally closed end and defining an opening leading to an interior chamber within the container body for holding a medical fluid; and
a pump device closing and sealing the opening in the container body and adapted to draw the medical fluid from the container body and deliver the medical fluid to a patient.

11. A fluid delivery system as claimed in claim 10 wherein the pump device comprises a gear pump.

12. A fluid delivery system as claimed in claim 11 wherein the gear pump is disposed in a housing sealing the opening in the container body.

13. A fluid delivery system as claimed in claim 10 further comprising a plunger disposed in the container body for dispensing the medical fluid from the container body.

14. A fluid delivery system as claimed in claim 10 further comprising an encoding device associated with the container body and operable to provide container body information to a sensor.

15. A fluid delivery system as claimed in claim 14 wherein the encoding device comprises an optically readable device.

16. A fluid delivery system as claimed in claim 14 wherein the container body information comprises at least container body volume information and the type of medical fluid contained in the container body.

17. A fluid delivery system as claimed in claim 10 wherein the container body and pump device are disposable as a singular unit.

18. A fluid delivery system as claimed in claim 10 further comprising a drive device operatively connected to the pump device for driving the pump device.

19. A fluid delivery system as claimed in claim 18 wherein the pump device comprises a gear pump and the drive device is adapted to drive the gear pump.

20. A fluid delivery system as claimed in claim 10 further comprising a fluid level sensor associated with the container body for determining the level of medical fluid in the container body.

21. A fluid delivery system as claimed in claim 20 wherein the fluid level sensor is adapted to sense when the medical fluid reaches a minimum level in the container body.

22. A fluid delivery system as claimed in claim 10 wherein the bulk container and pump assembly is releasably lockable to a base supporting a drive device adapted to drive the pump device.

23. A fluid delivery system as claimed in claim 22 wherein the bulk container and pump assembly is releasably lockable to the base with at least one releasable clamp.

24. A fluid delivery system as claimed in claim 23 further comprising a sensor associated with the releasable clamp and adapted to identify a locked position of the releasable clamp.

25. A fluid delivery system, comprising:
a control unit;
a drive device operatively controlled by the control unit; and
a bulk container and pump assembly in fluid communication, comprising:
a container body comprising a generally closed end and defining an opening leading to an interior chamber within the container body for holding a medical fluid; and
a pump device closing and sealing the opening in the container body and adapted to draw the medical fluid from the container body and deliver the medical fluid to a patient, wherein the drive device is operatively connected to a pump device for driving the pump device.

26. A fluid delivery system as claimed in claim 25 further comprising a fluid level sensor associated with the container body for determining the level of medical fluid in the container body.

27. A fluid delivery system as claimed in claim 26 wherein the fluid level sensor is adapted to sense when the medical fluid reaches a minimum level in the container body and send a signal to the control unit.

28. A fluid delivery system as claimed in claim 25 further comprising a sensor associated with a releasable clamp adapted to releasably lock the bulk container and pump assembly to a base supporting the drive device, and wherein the sensor is adapted to identify a locked position of the releasable clamp and send a signal to the control unit.

29. A fluid delivery system as claimed in claim 25 further comprising an encoding device associated with the container body and operable to provide container body information to a sensor operatively connected to the control device.

30. A fluid delivery system as claimed in claim 15 wherein the encoding device comprises an optically readable device.
